(19) United States
(12) Patent Application Publication
(10) Pub. No.: US 2012/0316610 A1
(43) Pub. Date: Dec. 13, 2012

(54) SYSTEMS AND METHODS FOR CUSTOMIZING STIMULATION USING IMPLANTABLE ELECTRICAL STIMULATION SYSTEMS

(75) Inventors: Anne Margaret Pianca, Santa Monica, CA (US); Dongchul Lee, Agua Dulce, CA (US)

(73) Assignee: Boston Scientific Neuromodulation Corporation, Valencia, CA (US)

(21) Appl. No.: 13/491,997

(22) Filed: Jun. 8, 2012

Related U.S. Application Data
(60) Provisional application No. 61/495,791, filed on Jun. 10, 2011.

(51) Int. Cl. A61N 1/05; A61N 1/36

(52) U.S. Cl. 607/2; 607/116

(57) ABSTRACT

An implantable paddle lead includes at least one lead body with a proximal end and a distal end. Terminals are disposed at the proximal end of the lead body. A paddle body is coupled to the distal end of the lead body. The paddle body has a length, a width, a first surface, and an opposing second surface. Electrodes are disposed on the first surface of the paddle body. At least one region of the paddle body has a higher pliability than remaining portions of the paddle body. The at least one region of higher pliability extends along the second surface of the paddle body. The paddle body is configured and arranged to preferentially bend along the at least one region of higher pliability. Conductors are disposed along the paddle lead. Each conductor electrically couples at least one of the electrodes to at least one of the terminals.

### CROSS-REFERENCE TO RELATED APPLICATIONS

This application claims the benefit under 35 U.S.C. §119(e) of U.S. Provisional Patent Application Ser. No. 61/495,791 filed on Jun. 10, 2011, which is incorporated herein by reference.

### FIELD

The present invention is directed to the area of implantable electrical stimulation systems and methods of making and using the systems. The present invention is also directed to electrical stimulation paddle leads having paddle bodies with regions of increased pliability for promoting customization of stimulation, as well as methods of making and using the paddle bodies, paddle leads, and electrical stimulation systems.

### BACKGROUND

Implantable electrical stimulation systems have proven therapeutic in a variety of diseases and disorders. For example, spinal cord stimulation systems have been used as a therapeutic modality for the treatment of chronic pain syndromes. Peripheral nerve stimulation has been used to treat incontinence, as well as a number of other applications under investigation. Functional electrical stimulation systems have been applied to restore some functionality to paralyzed extremities in spinal cord injury patients.

Stimulators have been developed to provide therapy for a variety of treatments. A stimulator can include a control module (with a pulse generator), one or more leads, and an array of stimulator electrodes on each lead. The stimulator electrodes are in contact with or near the nerves, muscles, or other tissue to be stimulated. The pulse generator in the control module generates electrical pulses that are delivered by the electrodes to body tissue.

### BRIEF SUMMARY

In one embodiment, an implantable paddle lead includes at least one lead body with a proximal end and a distal end. A plurality of terminals are disposed at the proximal end of the lead body. A paddle body is coupled to the distal end of the lead body. The paddle body has a length, a width, a first surface, and an opposing second surface. A plurality of electrodes are disposed on the first surface of the paddle body. At least one region of the paddle body has a higher pliability than remaining portions of the paddle body. The at least one region of higher pliability extends along the second surface of the paddle body. The paddle body is configured and arranged to preferentially bend along the at least one region of higher pliability. A plurality of conductors are disposed along the paddle lead. Each conductor electrically couples at least one of the electrodes to at least one of the terminals.

In another embodiment, an implantable paddle lead includes a plurality of lead bodies. Each of the plurality of lead bodes has a proximal end and a distal end. For each of the plurality of lead bodies a plurality of terminals are disposed at the proximal end of the lead body. A paddle body is coupled to the distal ends of each of the plurality of lead bodies. The paddle body has a length, a width, a first surface, and an opposing second surface. A plurality of electrodes are disposed on the first surface of the paddle body. At least one region of the paddle body has higher pliability than remaining portions of the paddle body. The at least one region of higher pliability extends between a first electrode of the plurality of electrodes and a second electrode of the plurality of electrodes. The paddle body is configured and arranged to separate along the at least one region of increased pliability. A plurality of conductors are disposed along the paddle lead. Each conductor electrically couples at least one of the electrodes to at least one of the terminals.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Detailed Description, which is to be read in association with the accompanying drawings, wherein:

FIG. 1 is a schematic view of one embodiment of an electrical stimulation system that includes a paddle body coupled to a control module via lead bodies, according to the invention;

FIG. 2A is a schematic side view of one embodiment of a plurality of connector assemblies disposed in the control module of FIG. 1, the connector assemblies configured and arranged to receive the proximal portions of the lead bodies of FIG. 1, according to the invention;

FIG. 2B is a schematic side view of one embodiment of a proximal portion of a lead body and a lead extension coupled to a control module, the lead extension configured and arranged to couple the proximal portion of the lead body to the control module, according to the invention;

FIG. 2C is a schematic side view of one embodiment of a connector assembly disposed in the control module of FIG. 2B, the connector assembly configured and arranged to receive the lead extension of FIG. 2B, according to the invention;

FIG. 3 is a schematic longitudinal cross-sectional view of one embodiment of one of the connector assemblies of FIG. 1, according to the invention.

FIG. 4 is a schematic perspective view a control module with a header that defines four ports, according to the invention;

FIG. 5A is a schematic top view of one embodiment of a paddle body with two columns of electrodes disposed on a first surface of the paddle body and a region of increased pliability extending along a second surface of the paddle body, opposite to the first surface, according to the invention;

FIG. 5B is a schematic transverse cross-sectional view of one embodiment of the paddle body of FIG. 5A, according to the invention;

FIG. 5C is a schematic transverse cross-sectional view of one embodiment of the paddle body of FIG. 5A, the paddle body bent along a region of increased pliability, according to the invention;

FIG. 6A is a schematic transverse cross-sectional view of the paddle body of FIG. 5A abutting a dura surrounding a spinal cord, the paddle body in a flat configuration, according to the invention;

FIG. 6B is a schematic transverse cross-sectional view of electrodes of the paddle body of FIG. 5A abutting a dura surrounding a spinal cord, the paddle body in a bent configuration and pressed against the dura to align electrodes on the paddle body with the dura without flattening the dura, according to the invention;

FIG. 7A is a schematic top view of one embodiment of a paddle body with four columns of electrodes disposed on a first surface of the paddle body and regions of increased pliability extending along a second surface of the paddle body, opposite to the first surface, according to the invention;

FIG. 7B is a schematic transverse cross-sectional view of one embodiment of the paddle body of FIG. 7A, according to the invention;

FIG. 8A is a schematic transverse cross-sectional view of electrodes of the paddle body of FIG. 7A abutting a dura surrounding a spinal cord, the paddle body in a flat configuration and pressed against the dura to align the electrodes with the dura, thereby causing a portion of the dura to flatten, according to the invention;

FIG. 8B is a schematic transverse cross-sectional view of electrodes of the paddle body of FIG. 7A abutting a dura surrounding a spinal cord, the paddle body in a bent configuration and pressed against the dura to align electrodes on the paddle body with the dura without flattening the dura, according to the invention;

FIG. 9 is a schematic transverse cross-sectional view of one embodiment of a paddle body with two columns of electrodes disposed on a first surface of the paddle body and a region of increased pliability extending along a second surface of the paddle body, opposite to the first surface, according to the invention;

FIG. 10 is a schematic transverse cross-sectional view of one embodiment of a paddle body with two columns of electrodes disposed on a first surface of the paddle body and a region of increased pliability extending between the columns on the first surface of the paddle body, according to the invention;

FIG. 11 is a schematic transverse cross-sectional view of one embodiment of a paddle body with a first region of increased pliability defined between two columns of electrodes disposed on a first surface of the paddle body, and a second region of increased pliability defined along a second surface of the paddle body, opposite to the first surface, according to the invention;

FIG. 12 is a schematic top view of one embodiment of a paddle body with three columns of electrodes disposed on a first surface of the paddle body and a plurality of regions of increased pliability extending along a second surface of the paddle body, opposite to the first surface, according to the invention;

FIG. 13 is a schematic top view of one embodiment of a paddle body with five columns of electrodes disposed on a first surface of the paddle body and a plurality of regions of increased pliability extending along a second surface of the paddle body, opposite to the first surface, according to the invention;

FIG. 14A is a schematic top view of one embodiment of a paddle body partially separated along a region of increased pliability extending along the paddle body, according to the invention;

FIG. 14B is a schematic top view of one embodiment of the paddle body of FIG. 14A, the paddle body completely separated along a region of increased pliability to form multiple discrete stimulation members, according to the invention;

FIG. 15A is a schematic top view of another embodiment of a paddle body partially separated along a region of increased pliability extending along the paddle body, according to the invention;

FIG. 15B is a schematic top view of one embodiment of the paddle body of FIG. 15A, the paddle body separated along one of several regions of increased pliability extending along the paddle body to form multiple discrete stimulation members, according to the invention;

FIG. 15C is a schematic top view of one embodiment of the paddle body of FIG. 15A, the paddle body separated along each of several regions of increased pliability extending along the paddle body to form multiple discrete stimulation members, according to the invention;

FIG. 16A is a schematic top view of one embodiment of the paddle body of FIG. 7A, the paddle body separated along one of several regions of increased pliability extending along the paddle body to form multiple discrete stimulation members, according to the invention;

FIG. 16B is a schematic top view of one embodiment of the paddle body of FIG. 7A, the paddle body separated along several different regions of increased pliability extending along the paddle body to form multiple discrete stimulation members, according to the invention;

FIG. 16C is a schematic top view of one embodiment of the paddle body of FIG. 7A, the paddle body separated along one of several regions of increased pliability extending along the paddle body to form multiple discrete stimulation members, according to the invention;

FIG. 16D is a schematic top view of one embodiment of the paddle body of FIG. 7A, the paddle body separated along several different regions of increased pliability extending along the paddle body to form multiple discrete stimulation members, according to the invention;

FIG. 17A is a schematic top view of another embodiment of a paddle body with four columns of electrodes disposed on a first surface of the paddle body and several regions of increased pliability extending along a second surface of the paddle body, opposite to the first surface, according to the invention;

FIG. 17B is a schematic top view of one embodiment of the paddle body of FIG. 17A, the paddle body separated along each of several different regions of increased pliability extending along the paddle body to form multiple discrete stimulation members, according to the invention;

FIG. 18A is a schematic top view of yet another embodiment of a paddle body with four columns of electrodes disposed on a first surface of the paddle body and several regions of increased pliability extending along a second surface of the paddle body, opposite to the first surface, according to the invention;

FIG. 18B is a schematic top view of one embodiment of the paddle body of FIG. 18A, the paddle body separated along each of several different regions of increased pliability extending along the paddle body to form multiple discrete stimulation members, according to the invention; and

FIG. 19 is a schematic overview of one embodiment of components of a stimulation system, including an electronic subassembly disposed within a control module, according to the invention.

### DETAILED DESCRIPTION

The present invention is directed to the area of implantable electrical stimulation systems and methods of making and using the systems. The present invention is also directed to electrical stimulation paddle leads having paddle bodies with regions of increased pliability for promoting customization of stimulation, as well as methods of making and using the paddle bodies, paddle leads, and electrical stimulation systems.

The regions of pliability incorporated with paddle lead embodiments can be used to conform the paddle to one or more curved portions of target tissue. In addition, the regions of pliability incorporated in the paddle lead embodiments may be used as guides to separate or cut an original paddle into two or more smaller, customized paddles. These smaller, customized, paddles may be placed on various portions of the target tissue and provide increased flexibility of paddle placement and thus placement of electrodes with the target tissue. As such, the paddle lead embodiments described may be used in the original state, with intact regions of pliability, to provide a conformable paddle lead; or the health practitioner may cut, or separate, the original paddle along one or more lines or regions of pliability to customize the numbers, or sizes, or both of each customized paddle. It may also be possible to have smaller paddles that have been customized by cutting a larger, original paddle, the smaller paddles having bendable lines or regions that may conform to curved tissue.

Suitable implantable electrical stimulation systems include, but are not limited to, an electrode lead (“lead”) with one or more electrodes disposed on a distal end of the lead and one or more terminals disposed on one or more proximal ends of the lead. Leads include, for example, percutaneous leads, paddle leads, and cuff leads. Examples of electrical stimulation systems with leads are found in, for example, U.S. Pat. Nos. 6,181,969; 6,516,227; 6,609,029; 6,609,032; 6,741,892; 7,244,150; 7,672,734; 7,761,165; 7,949,395; 7,974,706; and 8,175,710; and U.S. Patent Applications Publication Nos. 2005/0165465, 2007/0150036; and 2008/0071320, all of which are incorporated by reference.

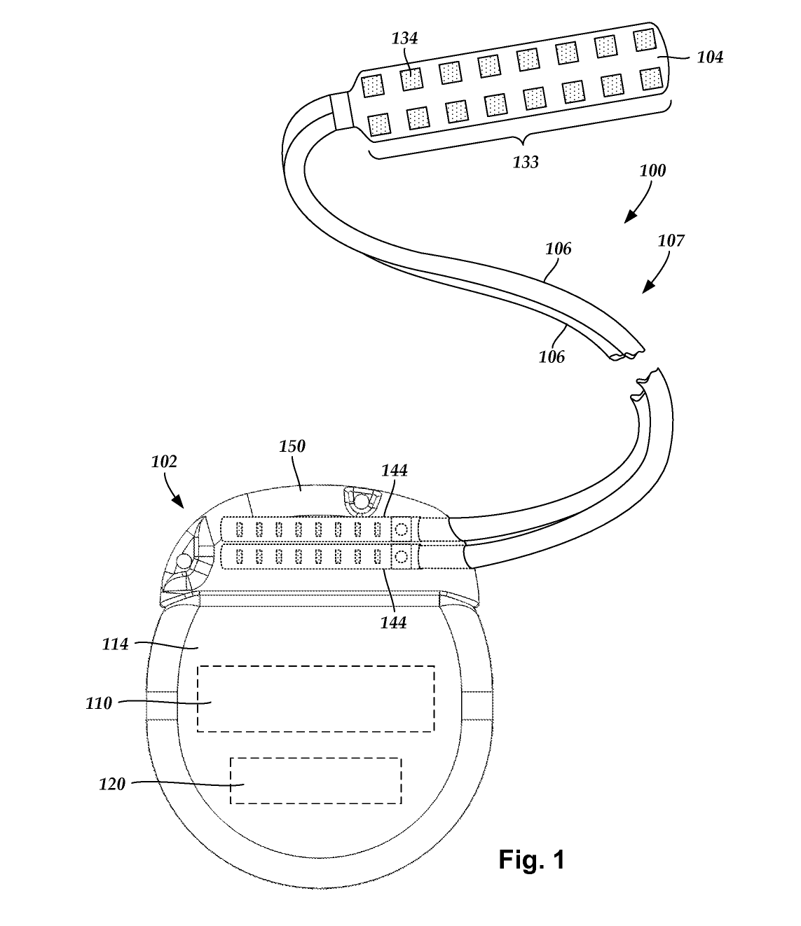

FIG. 1 illustrates schematically one embodiment of an electrical stimulation system 100. The electrical stimulation system includes a control module (e.g., a stimulator or pulse generator) 102, a paddle body 104, and one or more lead bodies 106 coupling the control module 102 to the paddle body 104. The paddle body 104 and the one or more lead bodies 106 collectively form a paddle lead 107. The paddle body 104 typically includes a plurality of electrodes 134 that form an array of electrodes 133. The control module 102 typically includes an electronic subassembly 110 and an optional power source 120 disposed in a sealed housing 114. In FIG. 1, two lead bodies 106 are shown coupled to the control module 102.

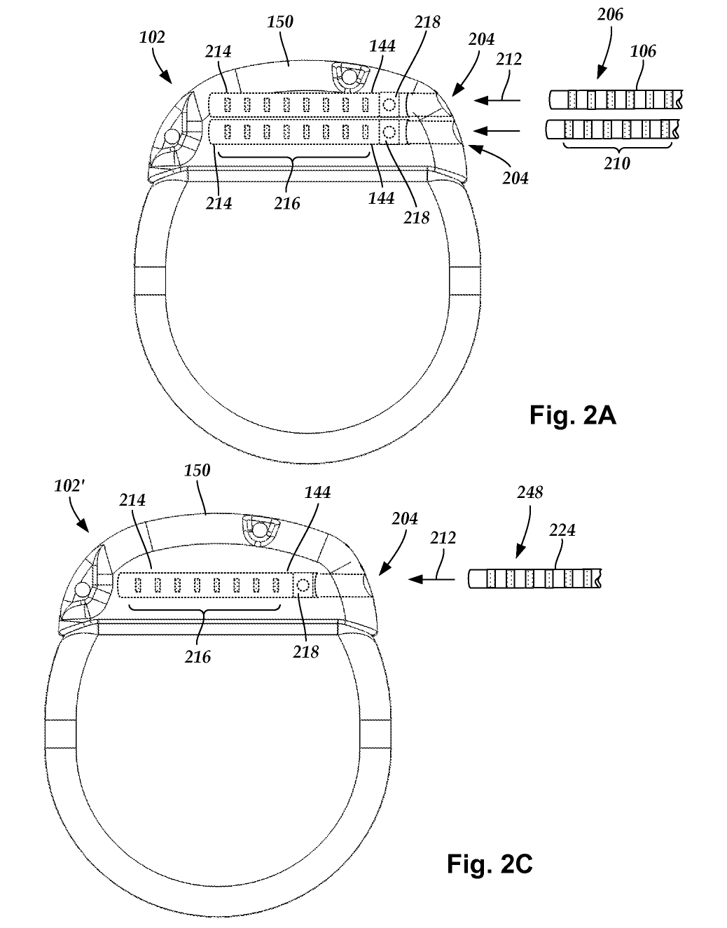

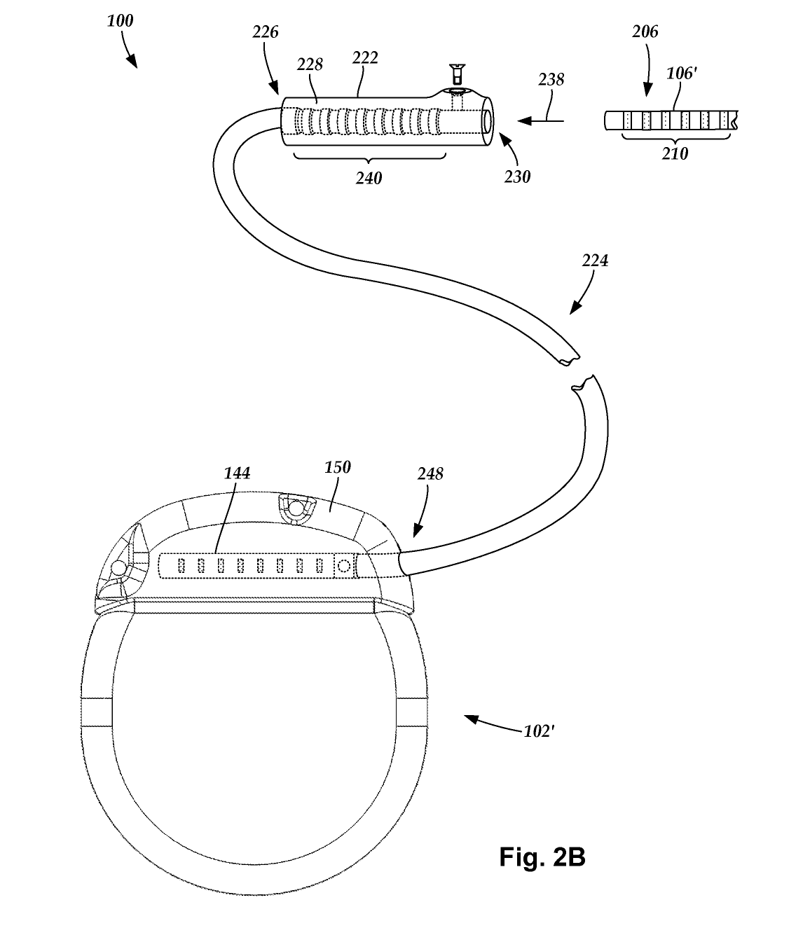

The control module 102 typically includes one or more connector assemblies 144 into which the proximal end of the one or more lead bodies 106 can be plugged to make an electrical connection via connector contacts (e.g., 216 in FIG. 2A). The connector contacts are coupled to the electronic subassembly 110 and the terminals are coupled to the electrodes 134. In FIG. 1, two connector assemblies 144 are shown.

The one or more connector assemblies 144 may be disposed in a header 150. The header 150 provides a protective covering over the one or more connector assemblies 144. The header 150 may be formed using any suitable process including, for example, casting, molding (including injection molding), and the like. In addition, one or more lead extensions 224 (see FIG. 2B) can be disposed between the one or more lead bodies 106 and the control module 102 to extend the distance between the one or more lead bodies 106 and the control module 102.

The electrical stimulation system or components of the electrical stimulation system, including one or more of the lead bodies 106, the paddle body 104, and the control module 102, are typically implanted into the body of a patient. The electrical stimulation system can be used for a variety of applications including, but not limited to, spinal cord stimulation, brain stimulation, neural stimulation, muscle activation via stimulation of nerves innervating muscle, and the like.

The electrodes 134 can be formed using any conductive, biocompatible material. Examples of suitable materials include metals, alloys, conductive polymers, conductive carbon, and the like, as well as combinations thereof. In at least some embodiments, one or more of the electrodes 134 are formed from one or more of: platinum, platinum iridium, palladium, palladium rhodium, or titanium.

The number of electrodes 134 in the array of electrodes 133 may vary. For example, there can be two, three, four, five, six, seven, eight, nine, ten, eleven, twelve, thirteen, fourteen, fifteen, sixteen, or more electrodes 134. As will be recognized, other numbers of electrodes 134 may also be used. In FIG. 1, sixteen electrodes 134 are shown. The electrodes 134 can be formed in any suitable shape including, for example, round, oval, triangular, rectangular, pentagonal, hexagonal, heptagonal, octagonal, or the like.

The electrodes of the paddle body 104 or one or more lead bodies 106 are typically disposed in, or separated by, a non-conductive, biocompatible material including, for example, silicone, polyurethane, and the like or combinations thereof. The paddle body 104 and one or more lead bodies 106 may be formed in the desired shape by any process including, for example, molding (including injection molding), casting, and the like. Electrodes and connecting wires can be disposed onto or within a paddle body either prior to or subsequent to a molding or casting process. The non-conductive material typically extends from the distal end of the lead body to the proximal end of each of the one or more lead bodies 106. The non-conductive, biocompatible material of the paddle body 104 and the one or more lead bodies 106 may be the same or different. The paddle body 104 and the one or more lead bodies 106 may be a unitary structure or can be formed as two separate structures that are permanently or detachably coupled together.

Terminals (e.g., 210 in FIG. 2A) are typically disposed at the proximal end of the one or more lead bodies 106 for connection to corresponding conductive contacts (e.g., 216 in FIG. 2A) in connector assemblies disposed on, for example, the control module 102 (or to other devices, such as conductive contacts on a lead extension, an operating room cable, a lead splitter, a lead adaptor, or the like). Conductive wires (not shown) extend from the terminals to the electrodes 134. Typically, one or more electrodes 134 are electrically coupled to a terminal (e.g., 210 in FIG. 2A). In some embodiments, each terminal (e.g., 210 in FIG. 2A) is only coupled to one electrode 134.

The conductive wires may be embedded in the non-conductive material of the paddle lead or can be disposed in one or more lumens (not shown) extending along the paddle lead. In some embodiments, there is an individual lumen for each conductive wire. In other embodiments, two or more conductive wires may extend through a lumen. There may also be one or more lumens (not shown) that open at, or near, the proximal end of the paddle lead, for example, for inserting a stylet wire to facilitate placement of the paddle lead within a body of a patient. Additionally, there may also be one or more lumens (not shown) that open at, or near, the distal end of the paddle lead, for example, for infusion of drugs or medication into the site of implantation of the paddle body 104. The one or more lumens may, optionally, be flushed continually, or on a regular basis, with saline, epidural fluid, or the like. The one or more lumens can be permanently or removably sealable at the distal end.

As discussed above, the one or more lead bodies 106 may be coupled to the one or more connector assemblies 144 disposed on the control module 102. The control module 102 can include any suitable number of connector assemblies 144 including, for example, two three, four, five, six, seven, eight, or more connector assemblies 144. It will be understood that other numbers of connector assemblies 144 may be used instead. In FIG. 1, each of the two lead bodies 106 includes eight terminals that are shown coupled with eight conductive contacts disposed in a different one of two different connector assemblies 144.

FIG. 2A is a schematic side view of one embodiment of the two lead bodies 106 shown in FIG. 1 configured and arranged for coupling with the control module 102. A plurality of connector assemblies 144 is disposed in the control module 102. In at least some embodiments, the control module 102 includes two, three, four, or more connector assemblies 144. Typically, the number of connector assemblies 144 disposed in the control module 102 is equal to the number of lead bodies 106 of the paddle lead. For example, in FIG. 2A, the two lead bodies 106 shown in FIG. 1 are shown configured and arranged for insertion into two connector assemblies 144 disposed on the control module 102.

The connector assemblies 144 each include a connector housing 214 and a plurality of connector contacts 216 disposed therein. Typically, the connector housing 214 defines a port (not shown) that provides access to the plurality of connector contacts 216. In at least some embodiments, the connector assemblies 144 further include retaining elements 218 configured and arranged to fasten the corresponding lead bodies 106 to the connector assemblies 144 when the lead bodies 106 are inserted into the connector assemblies 144 to prevent undesired detachment of the lead bodies 106 from the connector assemblies 144. For example, the retaining elements 218 may include apertures through which fasteners (e.g., set screws, pins, or the like) may be inserted and secured against an inserted lead body (or lead extension).

In FIG. 2A, the plurality of connector assemblies 144 are disposed in the header 150. In at least some embodiments, the header 150 defines one or more ports 204 into which a proximal end 206 of the one or more lead bodies 106 with terminals 210 can be inserted, as shown by directional arrows 212, in order to gain access to the connector contacts 216 disposed in the connector assemblies 144.

When the lead bodies 106 are inserted into the ports 204, the connector contacts 216 can be aligned with the terminals 210 disposed on the lead bodies 106 to electrically couple the control module 102 to the electrodes (134 of FIG. 1) disposed at a distal end of the lead bodies 106. Examples of connector assemblies in control modules are found in, for example, U.S. Pat. No. 7,244,150 and U.S. Patent Application Publication No. 2008/0071320, which are incorporated by reference.

In some instances, the electrical stimulation system may include one or more lead extensions. FIG. 2B is a schematic side view of one embodiment of a proximal end of a single lead body 106′ configured and arranged to couple with a lead extension 224 that is coupled with the control module 102′. In FIG. 2B, a lead extension connector assembly 222 is disposed at a distal end 226 of the lead extension 224. The lead extension connector assembly 222 includes a contact housing 228. The contact housing 228 defines at least one port 230 into which a proximal end 206 of the lead body 106′ with terminals 210 can be inserted, as shown by directional arrow 238. The lead extension connector assembly 222 also includes a plurality of connector contacts 240. When the lead body 106′ is inserted into the port 230, the connector contacts 240 disposed in the contact housing 228 can be aligned with the terminals 210 on the lead body 106 to electrically couple the lead extension 224 to electrodes (not shown) disposed on the lead body 106′.

The proximal end of a lead extension can be similarly configured and arranged as a proximal end of a lead body, such as one of the lead bodies 106, or the lead body 106′. The lead extension 224 may include a plurality of conductive wires (not shown) that electrically couple the connector contacts 240 to terminals at the proximal end 248 of the lead extension 224. The conductive wires disposed in the lead extension 224 can be electrically coupled to a plurality of terminals (not shown) disposed on the proximal end 248 of the lead extension 224.

FIG. 2C is a schematic side view of one embodiment of the lead extension 224 configured and arranged for coupling with the control module 102′. The control module 102′ includes a single connector assembly 144. Alternately, the control module 102′ may receive the lead body 106′ directly. It will be understood that the control modules 102 and 102′ can both receive either lead bodies or lead extensions. It will also be understood that the electrical stimulation system 100 can include a plurality of lead extensions 224. For example, each of the lead bodies 106 shown in FIGS. 1 and 2A can, alternatively, be coupled to a different lead extension 224 which, in turn, are each coupled to different ports of a two-port control module, such as the control module 102 of FIGS. 1 and 2A.

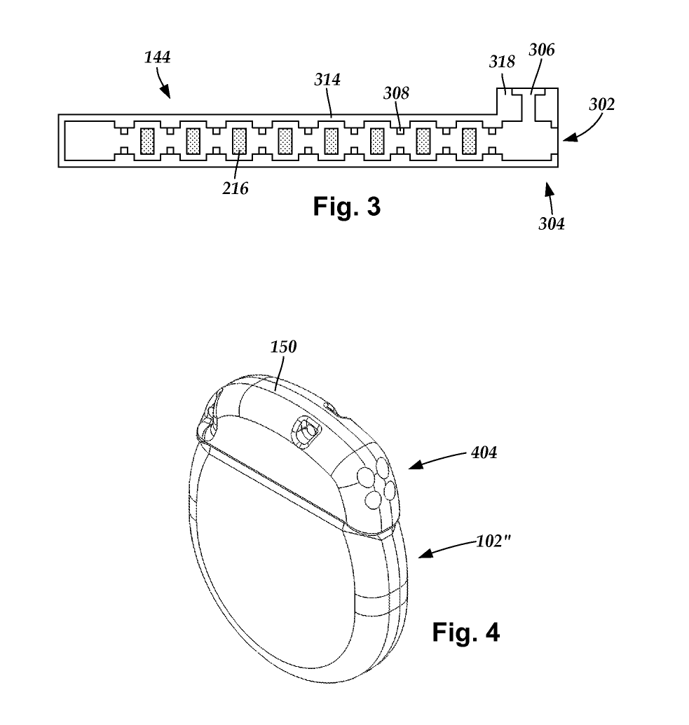

FIG. 3 is a schematic longitudinal cross-sectional view of one embodiment of one of the connector assemblies 144. The connector assembly 144 includes the connector housing 314 into which a lead body or lead extension can be inserted via a port 302 at a distal end 304 of the connector housing 314. In at least some embodiments, a retaining element 318 is coupled to the connector housing 314. The retaining element 318 defines an aperture 306 through which a fastener (e.g., a set screw, pin, or the like) may be inserted and secured against a lead body or lead extension when the lead body or lead extension is inserted into the port 302. Connector contacts, such as the connector contact 216, are disposed in the connector housing 314. In at least some embodiments, each of the connector assemblies 144 includes eight connector contacts.

The connector contacts 216 may be separated from one another by one or more non-conductive spacers (or seals), such as spacer 308, to prevent electrical contact between adjacent connector contacts 216. As discussed above, when a proximal end of a lead body or lead extension is inserted into the port 302, terminals disposed on the inserted lead body or lead extension align with the connector contacts 216, thereby establishing an electrical connection between the electronic subassembly 110 of the control module 102 and the electrodes 134 of the paddle body.

FIG. 4 is a schematic perspective view of a control module 102″. The header 150 of the control module 102″ defines four header ports 404. Collectively, the header ports 404 are configured and arranged to each receive one or more lead bodies 106 or one or more lead extensions (e.g., lead extension 224 of FIG. 2B), or both. The header 150 can define any suitable number of header ports 404 including, for example, one, two, three, four, five, six, seven, eight, or more header ports 404. In FIG. 4, the header 150 is shown defining four header ports 404. Thus, in at least some embodiments, the control module 102″ of FIG. 4 is configured and arranged to receive up to four lead bodies 106 or lead extensions 224, or a combination of both.

The header ports 404 can be defined in the header 150 in any suitable arrangement. In preferred embodiments, each of the header ports 404 are configured and arranged to align with one of the ports 302 of the one or more connector assemblies 144 disposed in the header 150. For example, in at least some embodiments, four connector assemblies 144 are disposed in the header 150 such that four header ports 404 defined in the header 150 align with the four ports 302 of the four connector assemblies 144. In at least some embodiments, the number of header ports 404 is no greater than the number of connector assemblies 144. In at least some embodiments, the number of header ports 404 is no less than the number of connector assemblies 144. In at least some embodiments, the number of header ports 404 is equal to the number of connector assemblies 144.

Patients undergoing electrical stimulation, such as spinal cord stimulation, represent a wide variety of conditions including, for example, chronic pain. In some cases, a single lead may not be able to sufficiently address the patient condition. This may especially be true for patients with disorders where pain may migrate over time, such as complex regional pain syndrome. In other cases, a lead may be larger, or provide more electrodes than are needed to provide therapy to a patient. Additionally, in some instances, a patient may have a feature, such as a build-up of scar tissue that obstructs one or more portions of a target stimulation location, thereby making it difficult to implant the lead in proximity to the target stimulation location. Accordingly, it may be advantageous to be able to customize stimulation on a patient-by-patient basis.

As herein described, a system and method for customizing electrical stimulation using a paddle lead is disclosed. The customizable electrical stimulation described herein enables versatility in at least one of the amount of electrodes, the physical arrangement of electrodes, or the physical arrangement of the paddle body used to provide therapy to the patient. In some cases, at least one of the shape or size of a paddle body can be altered. When the size of the paddle body is altered, the number of electrodes disposed on the paddle body may be changed, as well. Optionally, customization of the electrical stimulation system can be performed at the location of the implantation procedure by a medical practitioner. Thus, the customization can be performed during, or immediately prior, to an implantation procedure.

In some embodiments, the paddle body is selectively bendable to facilitate placement of the paddle body against a target stimulation location within a patient. Paddle bodies are often implanted into a patient such that the paddle bodies abut one or more curved body structures which receive electrical stimulation. For example, when a paddle body is used for spinal cord stimulation, the paddle body may be inserted into the patient's epidural space at a desired level of the spinal cord such that the paddle body is in proximity to the dura mater, or dura, which surrounds the spinal cord. As another example, the paddle body may be implanted in proximity to an anatomical structure which may have scar tissue built up along one or more portions of the anatomical structure.

At least some conventional paddle bodies are flat and formed from non-conductive materials that maintain a planar arrangement throughout the implanted lifetime of the paddle bodies. Unfortunately, disposing a flat paddle body into a curved space (e.g., an epidural space, over scar tissue, or the like) may cause the paddle body to flatten at least a portion of the anatomical structure to conform to the flat shape of the paddle body, or to align electrodes disposed on the paddle body to the patient tissue to be stimulated. Moreover, disposing a flat paddle body against a curved structure may create different propagation distances between different individual electrodes disposed on the planar paddle body and the stimulation target (e.g., a spinal cord) within the anatomical structure which the paddle body abuts. Creating different propagation distances between different individual electrodes disposed on a paddle body may reduce the efficacy of electrical stimulation.

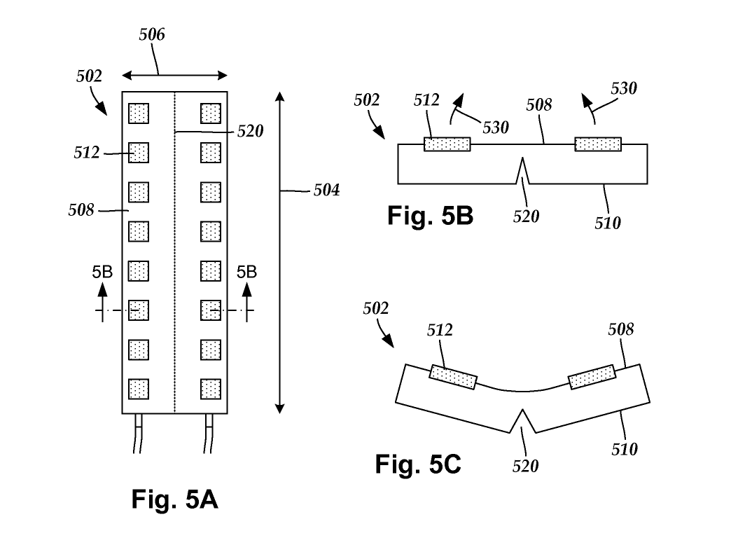

FIGS. 5A-18B illustrate several different embodiments of paddle bodies having one or more regions of the paddle body that have higher pliability than remaining regions of the paddle body (“regions of increased pliability”). The paddle bodies preferentially bend along the regions of increased pliability. In some cases, the paddle bodies can be adjustably bent along the regions of increased pliability. Bending the paddle bodies along the regions of increased pliability may improve stimulation by enabling the amplitude of stimulation to be lowered. In some cases, bending the paddle bodies along the regions of increased pliability may also decrease patient discomfort caused by the paddle slipping laterally within the epidural space. Moreover, bending the paddle bodies along the regions of increased pliability may decrease risk of causing damage to the target stimulation location by reducing the amount of distortion of the target stimulation location caused by the paddle body pressing against the target stimulation location post-implantation.

In some cases, the paddle bodies can be partially separated along the regions of increased pliability. Partially separated paddle bodies can be used to customize stimulation by changing the shape of the paddle body and by changing the center-to-center distances between at least some of the electrodes disposed on the paddle body. In some cases, the paddle bodies can be fully separated along the regions of increased pliability to form a plurality of discrete stimulation members. Fully-separating a paddle body into two or more discrete stimulation members can be used to customize stimulation by changing the center-to-center distances between at least some of the electrodes disposed on the paddle body, and also enabling target stimulation locations to be stimulated that would otherwise not be able to be stimulated by a paddle body because, for example, the target stimulation locations are too small to accommodate an entire paddle body.

FIG. 5A is a schematic top view of one embodiment of a paddle body 502 with a length 504, a width 506, a first surface 508, and a second surface 510 opposite to the first surface 508. FIG. 5B is a schematic transverse cross-sectional view of one embodiment of the paddle body 502.

Electrodes, such as electrode 512, are disposed on the first surface 508. The electrodes 512 can be arranged into any suitable configuration. In FIGS. 5A-5B, the electrodes 512 are arranged into columns extending along the length 504 of the paddle body 502, and also into rows extending along the width 506 of the paddle body 502. It will be understood that the electrode arrangements illustrated in FIGS. 5A-5B, as well as in other figures, are exemplary and are not meant to be limiting.

A region of increased pliability 520 extends along the second surface 510 of the paddle body 502. In FIGS. 5A and 5B, the region of increased pliability 520 extends along the length 504 of the paddle body 502 (i.e., in a direction that is parallel to the columns of electrodes 512, yet on the opposing surface of the paddle body 502 from the electrodes 512). Additionally, the region of increased pliability 520 is shown in FIGS. 5A and 5B as extending along the opposing side of the paddle body 502 from the electrodes 512 such that the region of increased pliability 520 extends between adjacent columns of electrodes 512. Thus, the region of increased pliability 520 extends along the paddle body 502 such that, were the paddle body 502 to be separated along the region of increased pliability 520, one of the two columns of electrodes 512 would be on one side of the region of increased pliability 520 and the other of the two columns of electrodes 512 would be on the other side of the region of increased pliability 520.

The region of increased pliability 520 may facilitate bending of the paddle body 502 along the region of increased pliability 520. FIG. 5B shows the paddle body 502 disposed in a substantially-straight position. The paddle body 502 can be adjustably bent along the region of increased pliability 520, as shown by arrows 530.

FIG. 5C is a schematic transverse cross-sectional view of one embodiment of the paddle body 502 bent along the region of increased pliability 520. As shown in FIG. 5C, the paddle body 502 is bent such that the first surface 508 forms a concave surface. Consequently, the electrodes 512 are inwardly-directed. In alternate embodiments, the regions of increased pliability 520 are configured to facilitate bending of the paddle body 502 such that the first surface 508 forms a convex surface and the electrodes 512 are outwardly-directed. In some cases, the regions of increased pliability 520 are configured to facilitate bending of the paddle body 502 such that the first surface 508 can be bent to form, a convex surface, a concave surface, or both (for example, when the paddle body 502 includes multiple regions of increased pliability).

The regions of increased pliability can be formed using the same material as the remaining regions of the paddle body. The one or more regions of increased pliability can include any suitable cut-out or weakened portion of the paddle body including, for example, one or more perforations, scores, notches, chamfers, clefts, grooves, indentations, depressions, gaps, rabbets, gashes, nicks, recesses, hinges (e.g., living hinges, or the like), or the like or combinations thereof. In some cases, the regions of increased pliability can be formed such that the regions of increased pliability are non-webbed.

In some cases, the paddle body may include a combination of one or more cut-out regions and one or more perforations extending along one or more portions of the cut-out regions. In which case, the perforations can be disposed within the cut-out regions such that the perforations are inset from other portions of an outer surface of the paddle body, thereby potentially increasing robustness of the paddle body. When the regions of increased pliability define one or more cut-out portions of the paddle body, the cut-out portions may extend at least 50%, 60%, 70%, 80%, or 90% of a thickness of the paddle body. In some cases, the cut-out portions may extend no more than 90% of the thickness of the paddle body. When the regions of increased pliability define one or more perforations, the one or more perforations may extend through the thickness of the paddle body 502 such that the perforations are defined along each of the two opposing surfaces 508, 510 of the paddle body 502.

The one or more regions of increased pliability can extend along the paddle body in any direction for any suitable length. The one or more regions of increased pliability can be either straight or curved. In some cases, one or more of the regions of increased pliability extend across an entire surface of the paddle body (e.g., across a width, length, or other axis of the paddle body). In other cases, one or more of the regions of increased pliability extend across at least 10%, 20%, 30%, 40%, 50%, 60%, 70%, 80%, or 90% of a surface of the paddle body.

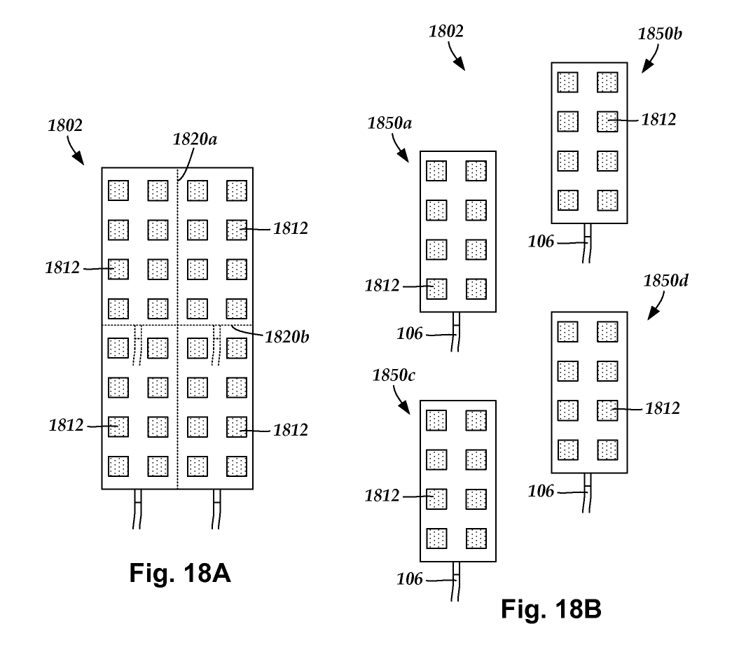

One or more regions of increased pliability can be extended in any suitable direction along the paddle body, as desired. For example, in some cases one or more regions of increased pliability can be extended across a length of the paddle body (see e.g., FIGS. 5A, 7A, and 12-16D). In other cases, one or more regions of increased pliability can be extended across a width of the paddle body in addition to, or in lieu of extending across the length of the paddle body (see e.g., FIGS. 17A-18B).

Any suitable number of regions of increased pliability can be extended along the paddle body. In some cases, the one or more regions of increased pliability extend between one or more sets of adjacent rows or columns of electrodes. The number of regions of increased pliability can be greater than, equal to, or less than a number of columns of electrodes. The number of regions of increased pliability can also be greater than, equal to, or less than a number of rows of electrodes. In some instances, the number of regions of increased pliability is equal to the number of columns of electrodes minus one (see e.g., FIG. 5A). In other instances, the number of regions of increased pliability is equal to the number of rows of electrodes minus one. When the one or more regions of increased pliability extend between adjacent electrodes, the regions of increased pliability may be sized to encompass as much of the distance between the adjacent electrodes as possible without compromising the structural integrity of the paddle body.

In the case of perforated regions of increased pliability, the regions of increased pliability may, optionally, include one or more materials disposed adjacent to the perforations that provide one or more mechanical benefits, as described below. The paddle body may include one or more reinforcement or insulation layers (e.g., polyethylene terephthalate, or the like) disposed adjacent to the perforations. The paddle body may include one or more materials that facilitate separation along the perforations. The paddle body may include one or more materials that reduce the risk of damaging other regions of the paddle body when the paddle body is separated along the perforations. The paddle body may include one or more materials that increase the consistency of separation of the paddle body along the perforations. The paddle body may include one or more materials that decrease the likelihood of undesired tearing along the paddle body.

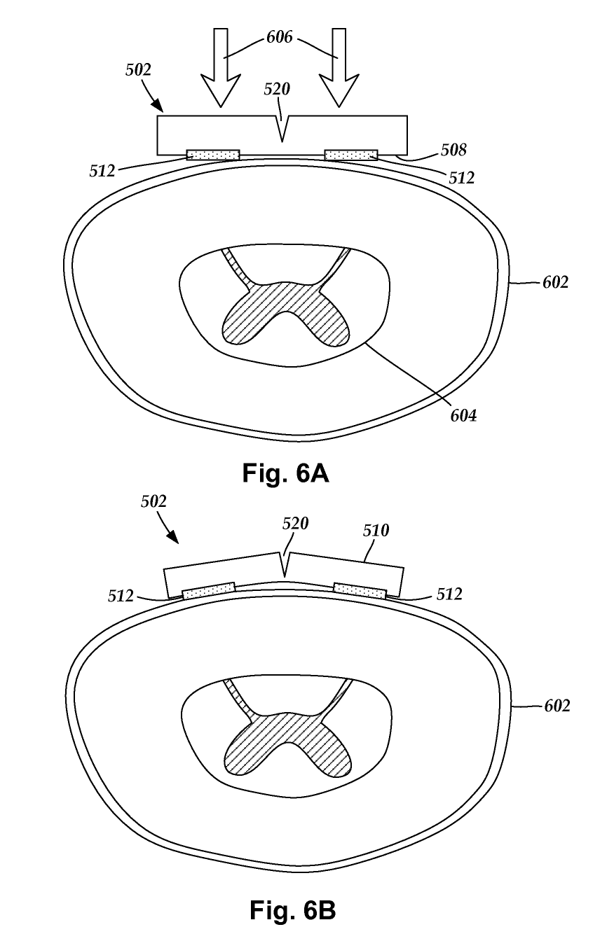

In at least some embodiments, the paddle body 502 may be implanted against one or more curved anatomical structures within a patient, such as against a portion of the patient's dura, in order to provide stimulation to the patient at one or more desired levels of the patient's spinal cord. FIGS. 6A-6B are schematic transverse cross-sectional views of the paddle body 502 positioned in an epidural space such that the first surface 508 of the paddle body 502 abuts a dura 602 surrounding a spinal cord 604. In FIG. 6A the paddle body 502 is in a flat configuration. When the paddle body 502 is in a flat configuration and positioned against a curved anatomical structure, the electrodes 512 may not align with the contours of the curved anatomical structure. For example, as shown in FIG. 6A, the electrodes 512 do not align with the contours of the dura 602.

In preferred embodiments, the one more regions of increased pliability 520 can enable the paddle body 502 to be adjustably bent to a shape conforming to the shape of the anatomical structure to which the paddle body 502 is to be implanted against prior to implantation. FIG. 6B is a schematic transverse cross-sectional view of the paddle body 502 bent along the region of increased pliability 520. The bend of the paddle body 502 corresponds to the natural curve of the dura 602 at the location where the paddle body 502 is implanted. Consequently, the electrodes 512 of the paddle body 502 align with the dura 602 without unnecessarily flattening the dura 602.

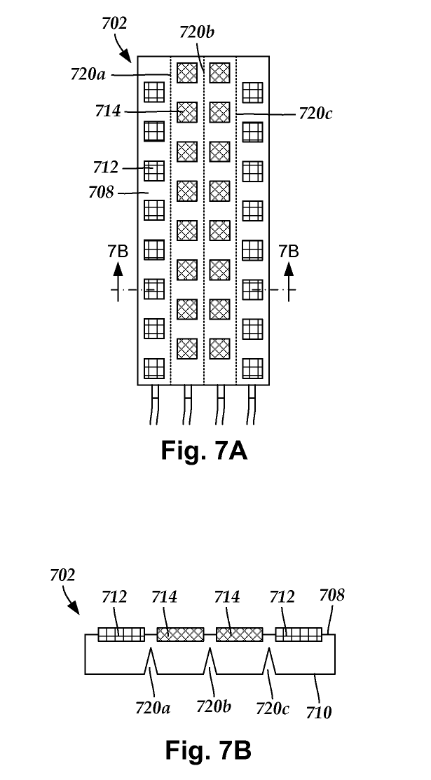

In FIGS. 5A-6B, the paddle body is shown with two columns of electrodes. It will be understood that the adjustable bending of the paddle body may similarly be performed with paddle bodies having any type of electrode arrangement (e.g., any suitable number of columns, number of rows, or the like). FIG. 7A is a schematic top view of one embodiment of a paddle body 702. FIG. 7B is a schematic transverse cross-sectional view of one embodiment of the paddle body 702. The paddle body 702 includes four columns of electrodes 712, 714 disposed on a first surface 708 of the paddle body 702 and a plurality of regions of increased pliability 720a, 720b, and 720c extending along a second surface 710 of the paddle body 702, opposite to the first surface 708.

In some instances, one or more of the electrodes 712, 714 may be used for monopolar stimulation. Optionally, one or more of the electrodes 712, 714 may be used for multipolar stimulation (e.g., tripolar, tetrapolar, or the like). In at least some embodiments, one or more of the electrodes may operate as either an anode 712 or a cathode 714.

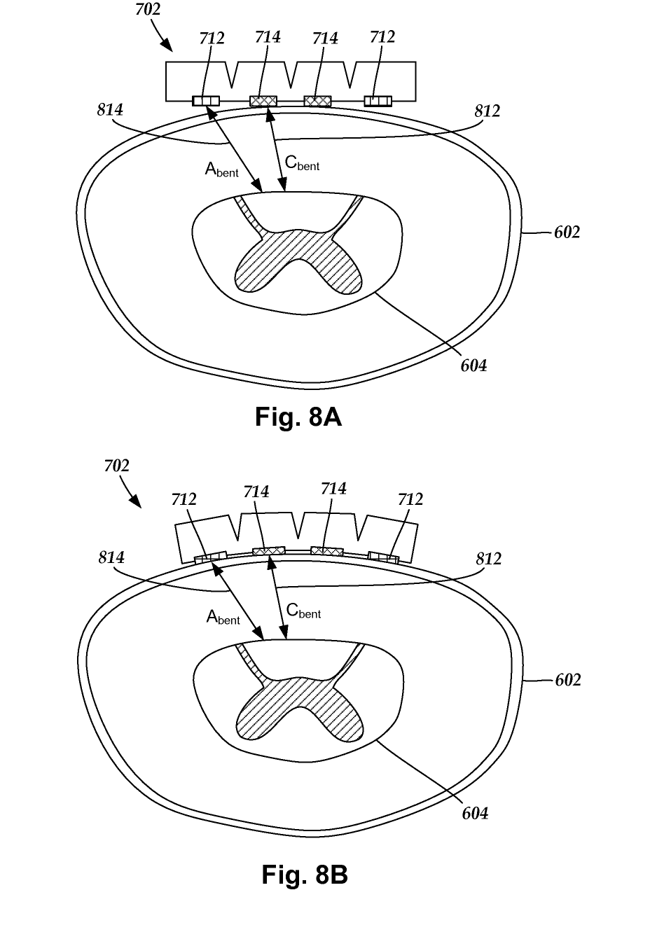

FIGS. 8A and 8B are schematic transverse cross-sectional views of the paddle body 702 abutting the dura 602 surrounding a spinal cord 604. In FIG. 8A the paddle body 702 is shown in a flat configuration and abutting the dura 602. In FIG. 8B, the paddle body 702 is shown in a bent configuration and abutting the dura 602. In FIG. 8A, the flat configuration of the paddle body 702 prevents at least some of the electrodes (e.g., electrodes 712) from physically contacting the dura 602. In contrast, the electrodes of the bent paddle body shown in FIG. 8B each physically contact the dura 602. Reducing, or eliminating, physical contact between the electrodes and the dura 602, as shown in FIG. 8A, may cause a corresponding reduction, or elimination, of electrical contact between the electrodes and the dura 602.

Using a bent paddle lead to provide electrical stimulation to a patient may also improve the efficacy of the electrical stimulation. For example, during transverse tripolar stimulation, when the paddle body 702 is in a flat configuration, as shown in FIG. 8A, the distance between the cathode 714 and the spinal cord 604 (shown in FIG. 8A by two-headed arrow 802) may be substantially less than the distance between a flanking anode 712 and the spinal cord (shown in FIG. 8A by two-headed arrow 804). Accordingly, the relatively close distance of the cathode 714 to the spinal cord 604, as compared to the anode 712, may reduce the amplitude of electrical stimulation. Consequently, during transverse tripolar stimulation the comparatively close distance from cathode 714 to the spinal cord 604 may attenuate the relative strength of electrical stimulation by the flanking anode 712.

In contrast, when, as shown in FIG. 8B, the paddle body 702 is bent to conform to the existing curve of the dura 602, the distance between the cathode 714 and the spinal cord 604 (shown in FIG. 8B by two-headed arrow 812) may be substantially similar to the distance between a flanking anode 712 and the spinal cord (shown in FIG. 8B by two-headed arrow 814), thereby increasing the relative strength (or the efficacy) of the flanking anode 712.

In FIGS. 5A-8B the regions of increased pliability are shown as elongated notches. It will be understood that the regions of increased pliability can be formed in any suitable manner. For example, the regions of increased pliability can be formed as one or more perforations, scores, notches, chamfers, clefts, grooves, indentations, depressions, gaps, rabbets, gashes, nicks, recesses, hinges (e.g., living hinges, or the like), or the like or combinations thereof.

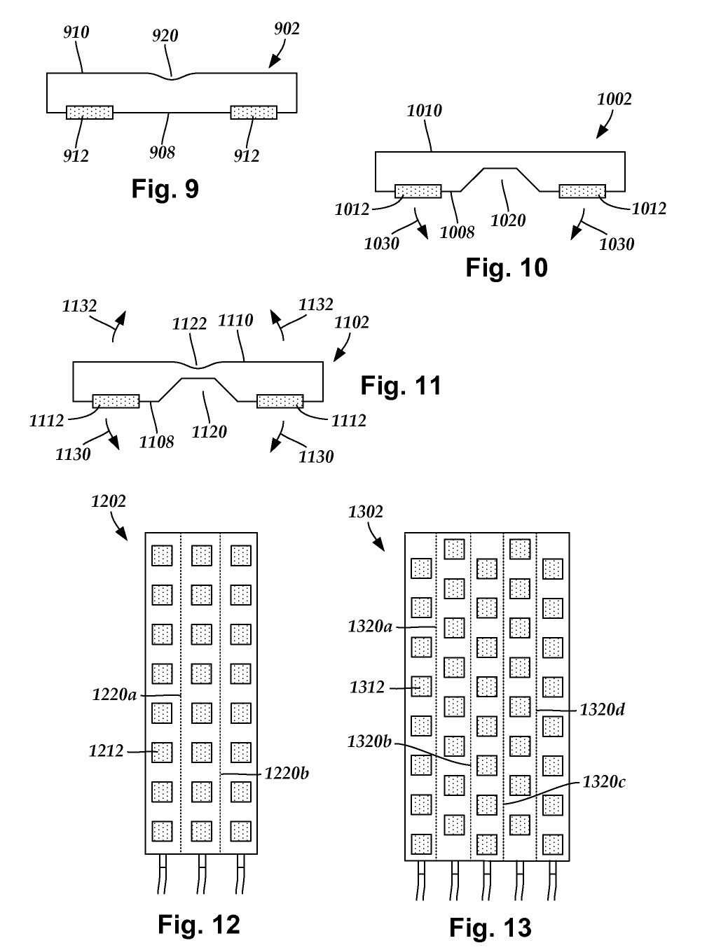

FIG. 9 is a schematic transverse cross-sectional view of another embodiment of a paddle body 902. A plurality of electrodes 912 is disposed on a first surface 908 of the paddle body 902. A region of increased pliability 920 extends along a second surface 910 of the paddle body 902, opposite to the first surface 908. In FIG. 9, the region of increased pliability 920 is shown as a depression.

In FIGS. 5A-9 the regions of increased pliability are shown extending along second surfaces of the paddle body, opposite to the first surface and the electrodes. It will be understood that the regions of increased pliability can extend along the first surface of the paddle body in lieu of, or in addition to, the second surface of the paddle body.

FIG. 10 is a schematic transverse cross-sectional view of yet another embodiment of a paddle body 1002. The paddle body 1002 has a first surface 1008 and a second surface 1010 opposite to the first surface 1008. A plurality of electrodes 1012 is disposed on a first surface 1008 of the paddle body 1002. A region of increased pliability 1020 extends along the first surface 1008 of the paddle body 1002. The region of increased pliability 1020 is shown preferentially facilitating bending of the paddle body 1002 in the directions shown by arrows 1030, thereby forming a concave first surface 1008. Consequently, the bending of the paddle body 1002 can cause the electrodes 1012 to be inwardly-directed.

FIG. 11 is a schematic transverse cross-sectional view of another embodiment of a paddle body 1102. The paddle body 1102 has a first surface 1108 and a second surface 1110 opposite to the first surface 1108. A plurality of electrodes 1112 is disposed on a first surface 1108 of the paddle body 1102. A first region of increased pliability 1120 extends along the first surface 1108 of the paddle body 1102 and a second region of increased pliability 1122 extends along the second surface 1110 of the paddle body 1102.

The first region of increased pliability 1120 is shown preferentially facilitating bending of the paddle body 1102 in the directions shown by arrows 1130, thereby forming a concave first surface 1108. Consequently, the bending of the paddle body 1102 can cause the electrodes 1112 on either side of the paddle body 1102 to turn inward. The second region of increased pliability 1122 is shown preferentially facilitating bending of the paddle body 1102 in the directions shown by arrows 1132, thereby forming a convex first surface 1108. Consequently, the bending of the paddle body 1102 can cause the electrodes 1112 to be outwardly-directed. Collectively, the first and second regions of increased pliability 1120 and 1122, respectively, enable the paddle body 1102 to bend, as desired, such that the electrodes 1112 are either turn inward or outward.

As mentioned above, the paddle body can have any suitable number of columns of electrodes. FIG. 12 is a schematic top view of one embodiment of a paddle body 1202 with three columns of electrodes 1212 disposed on the paddle body 1202. Regions of increased pliability 1220a and 1220b extend along portions of the paddle body 1202 such that each region of increased pliability 1220a and 1220b extends between at least two of the electrodes 1212.

FIG. 13 is a schematic top view of one embodiment of a paddle body 1302 with four columns of electrodes 1312 disposed on the paddle body 1302. Regions of increased pliability 1320a, 1320b, 1320c, and 1320d extend along portions of the paddle body 1202 such that each region of increased pliability 1320a-d extends between at least two of the electrodes 1212. As shown in FIGS. 12 and 13, when the electrodes 1212 and 1312, respectively, of a given column are arranged into columns, the electrodes 1212 and 1312, respectively, can be either longitudinally aligned or longitudinally offset from other columns of electrodes 1212 and 1312, respectively.

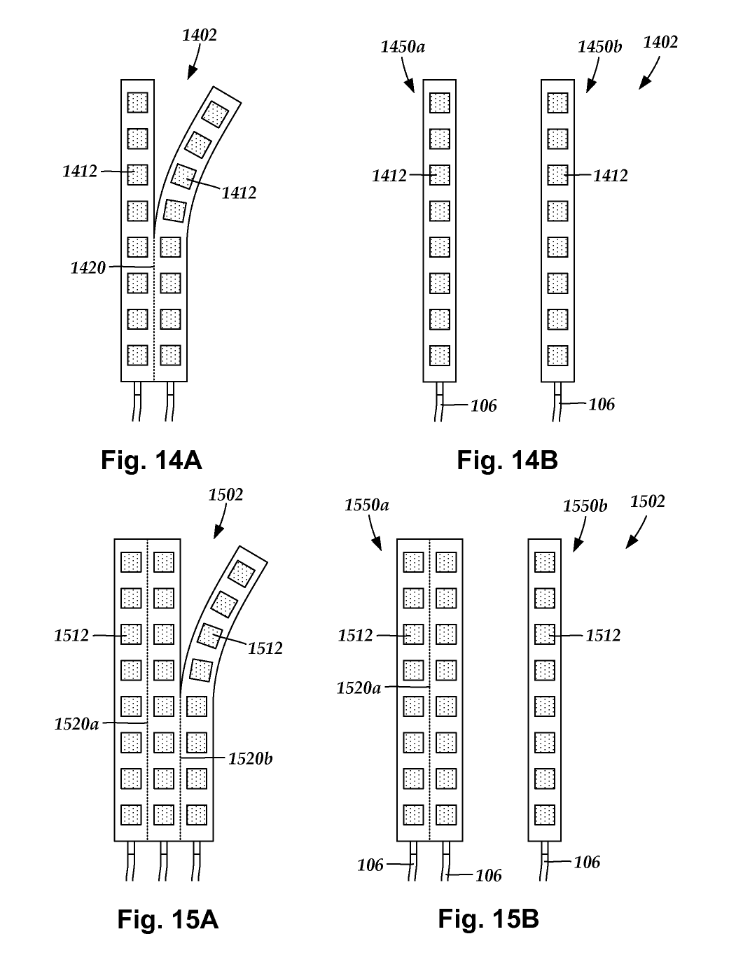

Turning to FIG. 14A, in at least some embodiments the paddle body can, optionally, be separated (if perforated) or cut using a cutting instrument along at least a portion of one of the regions (e.g., lines) of increased pliability. The regions of increased pliability enable the paddle body to be easily, and consistently, separated into a plurality of stimulation members, where each stimulation member includes at least one electrode. The one or more regions of increased pliability can be disposed on the paddle body in different numbers, or different arrangements, or both, to customize the stimulation received by the patient.

FIG. 14A is a schematic top view of one embodiment of a plurality of electrodes 1412 disposed on a paddle body 1402. A region of increased pliability 1420 extends along the paddle body 1402 such that the region of increased pliability 1420 extends between at least two of the electrodes 1412. The paddle body 1402 is partially separated along the region of increased pliability 1420. In some cases, for example when a patient has a build-up of scar tissue at a target stimulation location, the paddle body 1402 may be partially separated along the region of increased pliability 1420 such that one or more portions of the paddle body 1402 at least partially surround the scar tissue.

FIG. 14B is a schematic top view of one embodiment of the paddle body 1402 completely separated along the region of increased pliability 1420 into two discrete stimulation members 1450a and 1450b, each of the stimulation members 1450a and 1450b including at least one electrode 1412. Optionally, one or more of the stimulation members 1450a and 1450b can have separate lead bodies 106 for individually coupling with one or more connectors. In preferred embodiments, each of the stimulation members 1450a and 1450b has a separate lead body 106 for individually coupling with one or more connectors.

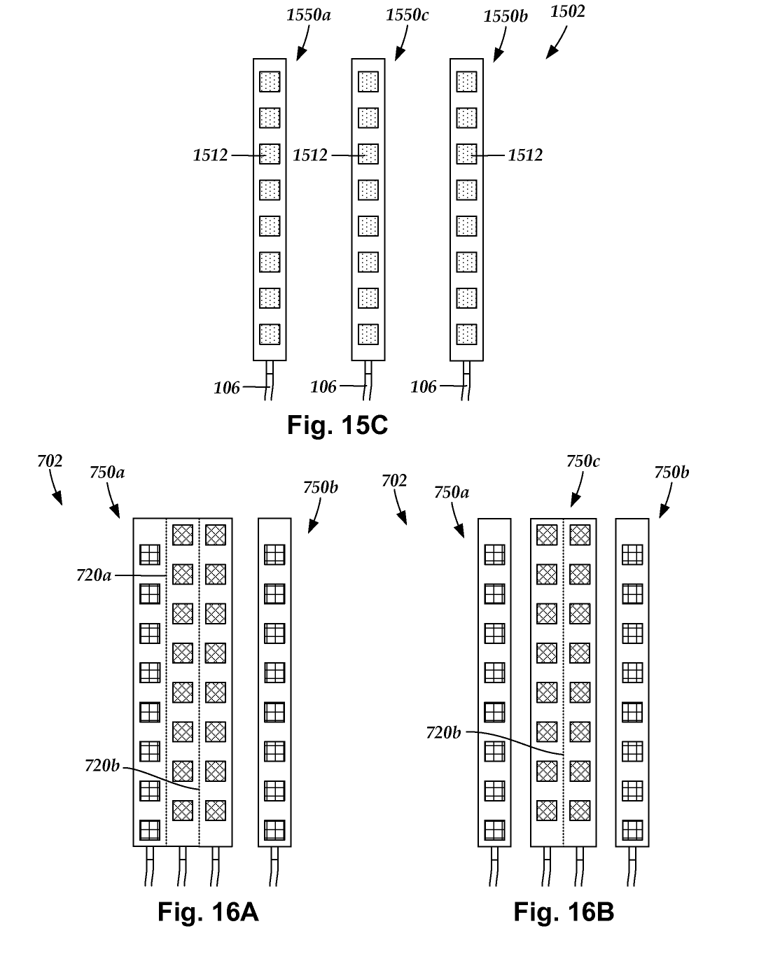

When the paddle body includes a plurality of regions of increased pliability, each of the regions of increased pliability can be left intact, partially separated, or completely separated or cut, as desired. FIG. 15A is a schematic top view of one embodiment of a plurality of electrodes 1512 disposed on a paddle body 1502. Two regions of increased pliability 1520a and 1520b extend along the paddle body 1502 such that each of the regions of increased pliability 1520a and 1520b extends between at least two of the electrodes 1512. The paddle body 1502 is partially separated along the region of increased pliability 1520b, while the region of increased pliability 1520a remains intact.

FIG. 15B is a schematic top view of one embodiment of the paddle body 1502 completely separated along the region of increased pliability 1520b such that the paddle body 1502 includes two discrete stimulation members 1550a and 1550b, each of the stimulation members 1550a and 1550b including at least one electrode 1512. The region of increased pliability 1520a remains intact. FIG. 15C is a schematic top view of one embodiment of the paddle body 1502 completely separated along both regions of increased pliability 1520a and 1520b such that the paddle body 1502 includes three discrete stimulation members 1550a, 1550b, and 1550c, each of the stimulation members 1550a, 1550b, and 1550c including at least one electrode 1512.

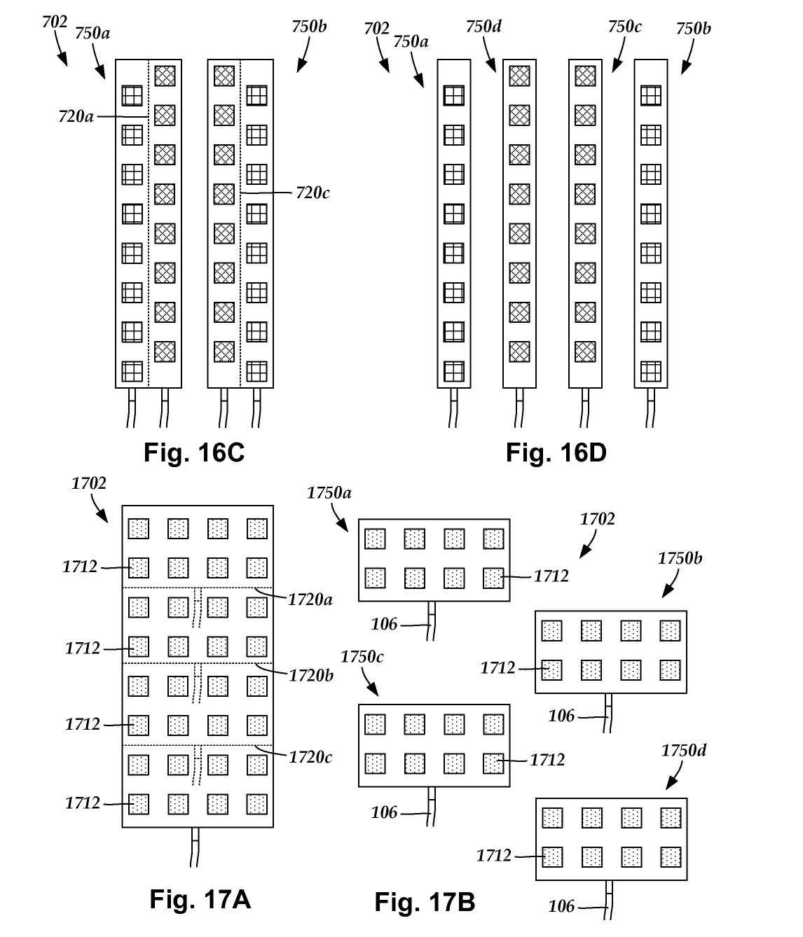

FIGS. 16A-16D illustrate different embodiments of the paddle body 702 being separated or cut along one or more of the three regions of increased pliability 720a, 720b, and 720c to form two or more discrete stimulation members. FIG. 16A is a schematic top view of one embodiment of the paddle body 702 separated along the region of increased pliability 720c to form stimulation members 750a and 750b. FIG. 16B is a schematic top view of one embodiment of the paddle body 702 separated along the regions of increased pliability 720a and 720c to form stimulation members 750a, 750b, and 750c. FIG. 16C is a schematic top view of one embodiment of the paddle body 702 separated or cut along the region of increased pliability 720b to form stimulation members 750a and 750b. FIG. 16D is a schematic top view of one embodiment of the paddle body 702 separated along the regions of increased pliability 720a, 720b, and 720c to form stimulation members 750a, 750b, 750c, and 750d.

In FIGS. 5A-16D the regions of increased pliability are shown extending between columns of electrodes. As discussed above, the regions of increased pliability can be disposed along the paddle body in any suitable direction. For example, in some cases one or more regions of increased pliability can be extended between rows of electrodes.

FIG. 17A is a schematic top view of another embodiment of a paddle body 1702. Electrodes 1712 are disposed on the paddle body 1702 such that the electrodes 1712 form rows. Regions of increased pliability 1720a, 1720b, and 1720c extend along the paddle body 1702 between at least two of the rows of electrodes 1712. FIG. 17B is a schematic top view of one embodiment of the paddle body 1702 separated along the regions of increased pliability 1720a, 1720b, and 1720c to form discrete stimulation members 1750a, 1750b, 1750c, and 1750d.

Optionally, one or more of the stimulation members 1750a, 1750b, 1750c, and 1750d can have separate lead bodies 106 for individually coupling with one or more connectors. In preferred embodiments, each of the stimulation members 1750a, 1750b, 1750c, and 1750d has a separate lead body 106 for individually coupling with one or more connectors.

In some cases, the paddle body includes one or more regions of increased pliability that extends between columns of electrodes, and one or more regions of increased pliability that extends between rows of electrodes. FIG. 18A is a schematic top view of yet another embodiment of a paddle body 1802. Electrodes 1812 are disposed on the paddle body 1802 such that the electrodes 1812 form one or more rows and one or more columns. A first region of increased pliability 1820a extends along the paddle body 1802 between at least two columns of electrodes 1812. A second region of increased pliability 1820b extends along the paddle body 1802 between at least two rows of electrodes 1812.

FIG. 18B is a schematic top view of one embodiment of the paddle body 1802 separated along the regions of increased pliability 1820a and 1820b to form discrete stimulation members 1850a, 1850b, 1850c, and 1850d. Optionally, one or more of the stimulation members 1850a, 1850b, 1850c, and 1850d can have separate lead bodies 106 for individually coupling with one or more connectors. In preferred embodiments, each of the stimulation members 1850a, 1850b, 1850c, and 1850d has a separate lead body 106 for individually coupling with one or more connectors.

In sum, the regions of pliability described herein enable the paddle body to be used as a conformable paddle and, optionally, as two or more customizable paddles cut or separated from the larger original paddle body. It will be understood that the two or more customized paddles may have additional regions of pliability that enable these smaller, customized paddles to also be conformable to curved body tissue.

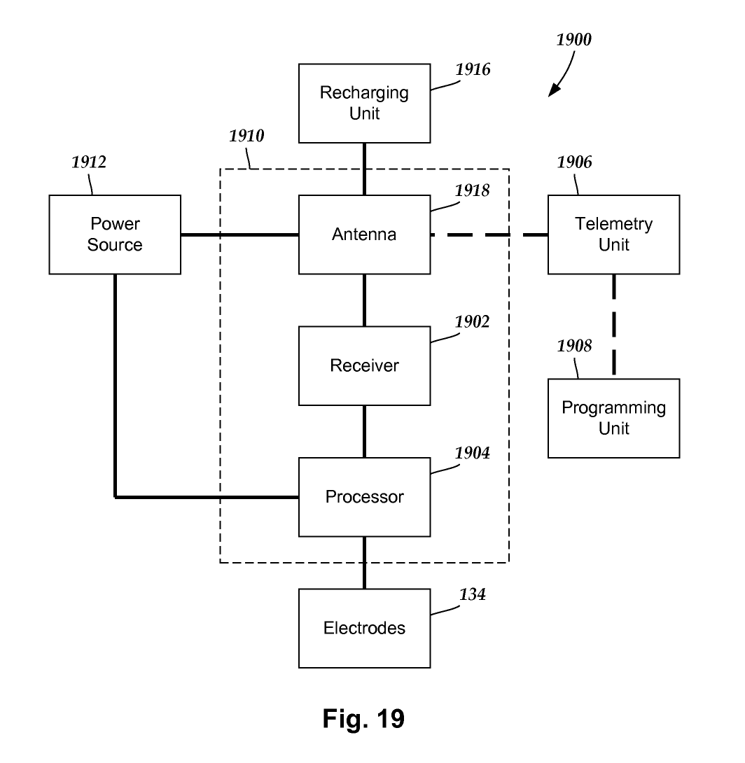

FIG. 19 is a schematic overview of one embodiment of components of an electrical stimulation system 1900 including an electronic subassembly 1910 disposed within a control module. It will be understood that the electrical stimulation system can include more, fewer, or different components and can have a variety of different configurations including those configurations disclosed in the stimulator references cited herein.

Some of the components (for example, power source 1912, antenna 1918, receiver 1902, and processor 1904) of the electrical stimulation system can be positioned on one or more circuit boards or similar carriers within a sealed housing of an implantable pulse generator, if desired. Any power source 1912 can be used including, for example, a battery such as a primary battery or a rechargeable battery. Examples of other power sources include super capacitors, nuclear or atomic batteries, mechanical resonators, infrared collectors, thermally-powered energy sources, flexural powered energy sources, bioenergy power sources, fuel cells, bioelectric cells, osmotic pressure pumps, and the like including the power sources described in U.S. Pat. No. 7,437,193, incorporated herein by reference.

As another alternative, power can be supplied by an external power source through inductive coupling via the optional antenna 1918 or a secondary antenna. The external power source can be in a device that is mounted on the skin of the user or in a unit that is provided near the user on a permanent or periodic basis.

If the power source 1912 is a rechargeable battery, the battery may be recharged using the optional antenna 1918, if desired. Power can be provided to the battery for recharging by inductively coupling the battery through the antenna to a recharging unit 1916 external to the user. Examples of such arrangements can be found in the references identified above.

In one embodiment, electrical current is emitted by the electrodes 134 on the paddle or lead body to stimulate nerve fibers, muscle fibers, or other body tissues near the electrical stimulation system. A processor 1904 is generally included to control the timing and electrical characteristics of the electrical stimulation system. For example, the processor 1904 can, if desired, control one or more of the timing, frequency, strength, duration, and waveform of the pulses. In addition, the processor 1904 can select which electrodes can be used to provide stimulation, if desired. In some embodiments, the processor 1904 may select which electrode(s) are cathodes and which electrode(s) are anodes. In some embodiments, the processor 1904 may be used to identify which electrodes provide the most useful stimulation of the desired tissue.

Any processor can be used and can be as simple as an electronic device that, for example, produces pulses at a regular interval or the processor can be capable of receiving and interpreting instructions from an external programming unit 1908 that, for example, allows modification of pulse characteristics. In the illustrated embodiment, the processor 1904 is coupled to a receiver 1902 which, in turn, is coupled to the optional antenna 1918. This allows the processor 1904 to receive instructions from an external source to, for example, direct the pulse characteristics and the selection of electrodes, if desired.

In one embodiment, the antenna 1918 is capable of receiving signals (e.g., RF signals) from an external telemetry unit 1906 which is programmed by a programming unit 1908. The programming unit 1908 can be external to, or part of, the telemetry unit 1906. The telemetry unit 1906 can be a device that is worn on the skin of the user or can be carried by the user and can have a form similar to a pager, cellular phone, or remote control, if desired. As another alternative, the telemetry unit 1906 may not be worn or carried by the user but may only be available at a home station or at a clinician's office. The programming unit 1908 can be any unit that can provide information to the telemetry unit 1906 for transmission to the electrical stimulation system 1900. The programming unit 1908 can be part of the telemetry unit 1906 or can provide signals or information to the telemetry unit 1906 via a wireless or wired connection. One example of a suitable programming unit is a computer operated by the user or clinician to send signals to the telemetry unit 1906.

The signals sent to the processor 1904 via the antenna 1918 and receiver 1902 can be used to modify or otherwise direct the operation of the electrical stimulation system. For example, the signals may be used to modify the pulses of the electrical stimulation system such as modifying one or more of pulse duration, pulse frequency, pulse waveform, and pulse strength. The signals may also direct the electrical stimulation system 1900 to cease operation, to start operation, to start charging the battery, or to stop charging the battery. In other embodiments, the stimulation system does not include an antenna 1918 or receiver 1902 and the processor 1904 operates as programmed.

Optionally, the electrical stimulation system 1900 may include a transmitter (not shown) coupled to the processor 1904 and the antenna 1918 for transmitting signals back to the telemetry unit 1906 or another unit capable of receiving the signals. For example, the electrical stimulation system 1900 may transmit signals indicating whether the electrical stimulation system 1900 is operating properly or not or indicating when the battery needs to be charged or the level of charge remaining in the battery. The processor 1904 may also be capable of transmitting information about the pulse characteristics so that a user or clinician can determine or verify the characteristics.

The above specification, examples and data provide a description of the manufacture and use of the composition of the invention. Since many embodiments of the invention can be made without departing from the spirit and scope of the invention, the invention also resides in the claims hereinafter appended.

What is claimed as new and desired to be protected by Letters Patent of the United States is:

1. An implantable paddle lead comprising:
at least one lead body with a proximal end and a distal end;
a plurality of terminals disposed at the proximal end of the lead body;
a paddle body coupled to the distal end of the lead body, the paddle body having a length, a width, a first surface, and an opposing second surface;
a plurality of electrodes disposed on the first surface of the paddle body;
at least one region of the paddle body having higher pliability than adjacent portions of the paddle body, wherein the at least one region of higher pliability extends along the second surface of the paddle body, wherein the paddle body is configured and arranged to preferentially bend along the at least one region of higher pliability; and
a plurality of conductors disposed along the paddle lead, each conductor electrically coupling at least one of the electrodes to at least one of the terminals.

2. The paddle lead of claim 1, wherein the at least one region of higher pliability additionally extends along the first surface of the paddle body.

3. The paddle lead of claim 1, wherein the at least one region of higher pliability extends across one of the entire length or the entire width of the paddle body.

4. The paddle lead of claim 1, wherein the at least one region of higher pliability defines an elongated perforation.

5. The paddle lead of claim 1, wherein the at least one region of higher pliability defines an elongated line of higher pliability incorporated into the paddle body, the elongated line severable with a cutting instrument.

6. The paddle lead of claim 1, wherein the paddle body further comprises reinforcing material disposed adjacent to the at least one region of higher pliability.

7. The paddle lead of claim 1, wherein the paddle body is configured and arranged for permitting a user to at least partially separate portions of the paddle body from each other along the at least one region of higher pliability.

8. The paddle lead of claim 1, wherein the paddle body is configured and arranged for permitting a user to completely separate portions of the paddle body from each other along the at least one region of higher pliability to yield at least two smaller-sized paddle bodies.

9. An implantable paddle lead comprising:
a plurality of lead bodies, each of the plurality of lead bodes having a proximal end and a distal end;
for each of the plurality of lead bodies a plurality of terminals disposed at the proximal end of the lead body;
a paddle body coupled to the distal ends of each of the plurality of lead bodies, the paddle body having a length, a width, a first surface, and an opposing second surface;
a plurality of electrodes disposed on the first surface of the paddle body;
at least one region of the paddle body having higher pliability than adjacent portions of the paddle body, wherein the at least one region of higher pliability extends between a first electrode of the plurality of electrodes and a second electrode of the plurality of electrodes, wherein the paddle body is configured and arranged to separate along the at least one region of higher pliability; and
a plurality of conductors disposed along the paddle lead, each conductor electrically coupling at least one of the electrodes to at least one of the terminals.

10. The paddle lead of claim 9, wherein the paddle body is configured and arranged for permitting a user to partially separate portions of the paddle body from each other along the at least one region of higher pliability, which region includes a perforation to facilitate bending or cutting of the region.

11. The paddle lead of claim 9, wherein the paddle body is configured and arranged for permitting a user to completely separate portions of the paddle body from each other along the at least one region of higher pliability such that the paddle body separates into a first stimulation member and a second stimulation member.

12. The paddle lead of claim 11, wherein the first electrode is disposed on the first stimulation member and the second electrode is disposed on the second stimulation member.

13. The paddle lead of claim 12, wherein a first lead body of the plurality of lead bodies is coupled to the first stimulation member and a second lead body of the plurality of lead bodies is coupled to the second stimulation member.

14. The paddle lead of claim 9, wherein the at least one perforation extends along the length of the paddle body.

15. The paddle lead of claim 9, wherein the at least one perforation extends along the width of the paddle body.

16. The paddle lead of claim 9, wherein the at least one perforation comprises a first perforation and a second perforation, and wherein the first perforation extends along the length of the paddle body and the second perforation extends along the width of the paddle body.

17. An electrical stimulation system comprising
the paddle lead of claim 9;
a control module configured and arranged to electrically couple to the proximal end of the lead body, the control module comprising
a housing, and
an electronic subassembly disposed in the housing; and
at least one connector for receiving the at least one lead body, the at least one connector having a proximal end, a distal end, and a longitudinal length, the at least one connector configured and arranged to receive the at least one lead body, the at least one connector comprising
a connector housing defining a port at the distal end of the connector, the port configured and arranged for receiving the proximal end of the at least one lead body, and
a plurality of connector contacts disposed in the connector housing, the connector contacts configured and arranged to couple to at least one of the plurality of terminals disposed on the proximal end of the at least one lead body.

18. A method for implanting a paddle lead into a patient, the method comprising:
providing the electrical stimulation system of claim 1;
inserting the paddle lead into the patient such that the first surface of the paddle body abuts an anatomical structure with a curved surface; and
bending the paddle body along the at least one region of higher pliability such that the first surface of the paddle body conforms to a shape of the curved surface of the anatomical structure.

19. The method of claim 18, further comprising partially separating portions of the paddle body from each other along the at least one region of higher pliability.

20. A method for implanting a paddle lead into a patient, the method comprising:
providing the electrical stimulation system of claim 1;
cutting or separating the paddle lead into at least two paddle bodies; and
inserting the at least two paddle bodies into a patient.
